SEQUENCE LISTING (12 sequences)

SEQ ID NO: 1 - DNA, 538 bp, Homo sapiens
tcttttctcaaagttcctgccttgctagactgttagctctttgaggacagggactatgtcttatcaatcactattattttcctgttacctagcatgggacaagtacacaacacatatttgttcaatgaatgaatgaatgtcttctaaaagactcctctgattgggaggacaatatctataattgggatgtgaatcatttcttcagtggaataagagcacaacggcacaaccttcaaggacatattatctactatgaacattttactgtgagactctttattttgccttctacttgcgctgaaatgaaaccaaaacaggccgttgggttcacaagtcaatatatgttggatgaggattctgttgccttattggggactgtgagacttatctggtatgagaagccagtaataaacctttgacctgttttaaccaatgaagattaggaatatgttaatatgatgtaaattgctatttaagtgtaaagcagttccaagttttagtattcgggggattggtttatgataatttttcccctttg

SEQ ID NO: 2 - DNA, 3794 bp, Homo sapiens
ccaagttctacctcatgtttggaggatcttgctagctatggccctcgtactcggctccctgttgctgctggggctgtgcgggaactccttttcaggagggcagccttcatccacagatgctcctaaggcttggaattatgaattgcctgcaacaaattatgagacccaagactcccataaagctggacccattggcattctctttgaactagtgcatatctttctctatgtggtacagccgcgtgatttcccagaagatactttgagaaaattcttacagaaggcatatgaatccaaaattgattatgacaagccagaaactgtaatcttaggtctaaagattgtctactatgaagcagggattattctatgctgtgtcctggggctgctgtttattattctgatgcctctggtggggtatttcttttgtatgtgtcgttgctgtaacaaatgtggtggagaaatgcaccagcgacagaaggaaaatgggcccttcctgaggaaatgctttgcaatctccctgttggtgatttgtataataataagcattggcatcttctatggttttgtggcaaatcaccaggtaagaacccggatcaaaaggagtcggaaactggcagatagcaatttcaaggacttgcgaactctcttgaatgaaactccagagcaaatcaaatatatattggcccagtacaacactaccaaggacaaggcgttcacagatctgaacagtatcaattcagtgctaggaggcggaattcttgaccgactgagacccaacatcatccctgttcttgatgagattaagtccatggcaacagcgatcaaggagaccaaagaggcgttggagaacatgaacagcaccttgaagagcttgcaccaacaaagtacacagcttagcagcagtctgaccagcgtgaaaactagcctgcggtcatctctcaatgaccctctgtgcttggtgcatccatcaagtgaaacctgcaacagcatcagattgtctctaagccagctgaatagcaaccctgaactgaggcagcttccacccgtggatgcagaacttgacaacgttaataacgttcttaggacagatttggatggcctggtccaacagggctatcaatcccttaatgatatacctgacagagtacaacgccaaaccacgactgtcgtagcaggtatcaaaagggtcttgaattccattggttcagatatcgacaatgtaactcagcgtcttcctattcaggatatactctcagcattctctgtttatgttaataacactgaaagttacatccacagaaatttacctacattggaagagtatgattcatactggtggctgggtggcctggtcatctgctctctgctgaccctcatcgtgattttttactacctgggcttactgtgtggcgtgtgcggctatgacaggcatgccaccccgaccacccgaggctgtgtctccaacaccggaggcgtcttcctcatggttggagttggattaagtttcctcttttgctggatattgatgatcattgtggttcttacctttgtctttggtgcaaatgtggaaaaactgatctgtgaaccttacacgagcaaggaattattccgggttttggatacaccctacttactaaatgaagactgggaatactatctctctgggaagctatttaataaatcaaaaatgaagctcacttttgaacaagtttacagtgactgcaaaaaaaatagaggcacttacggcactcttcacctgcagaacagcttcaatatcagtgaacatctcaacattaatgagcatactggaagcataagcagtgaattggaaagtctgaaggtaaatcttaatatctttctgttgggtgcagcaggaagaaaaaaccttcaggattttgctgcttgtggaatagacagaatgaattatgacagctacttggctcagactggtaaatcccccgcaggagtgaatcttttatcatttgcatatgatctagaagcaaaagcaaacagtttgcccccaggaaatttgaggaactccctgaaaagagatgcacaaactattaaaacaattcaccagcaacgagtccttcctatagaacaatcactgagcactctataccaaagcgtcaagatacttcaacgcacagggaatggattgttggagagagtaactaggattctagcttctctggattttgctcagaacttcatcacaaacaatacttcctctgttattattgaggaaactaagaagtatgggagaacaataataggatattttgaacattatctgcagtggatcgagttctctatcagtgagaaagtggcatcgtgcaaacctgtggccaccgctctagatactgctgttgatgtctttctgtgtagctacattatcgaccccttgaatttgttttggtttggcataggaaaagctactgtatttttacttccggctctaatttttgcggtaaaactggctaagtactatcgtcgaatggattcggaggacgtgtacgatgatgttgaaactatacccatgaaaaatatggaaaatggtaataatggttatcataaagatcatgtatatggtattcacaatcctgttatgacaagcccatcacaacattgatagctgatgttgaaactgcttgagcatcaggatactcaaagtggaaaggatcacagatttttggtagtttctgggtctacaaggactttccaaatccaggagcaacgccagtggcaacgtagtgactcaggcgggcaccaaggcaacggcaccattggtctctgggtagtgctttaagaatgaacacaatcacgttatagtccatggtccatcactattcaaggatgactccctcccttcctgtctatttttgttttttacttttttacactgagtttctatttagacactacaacatatggggtgtttgttcccattggatgcatttctatcaaaactctatcaaatgtgatggctagattctaacatattgccatgtgtggagtgtgctgaacacacaccagtttacaggaaagatgcattttgtgtacagtaaacggtgtatataccttttgttaccacagagttttttaaacaaatgagtattataggactttcttctaaatgagctaaataagtcaccattgacttcttggtgctgttgaaaataatccattttcactaaaagtgtgtgaaacctacagcatattcttcacgcagagattttcatctattatactttatcaaagattggccatgttccacttggaaatggcatgcaaaagccatcatagagaaacctgcgtaactccatctgacaaattcaaaagagagagagagatcttgagagagaaatgctgttcgttcaaaagtggagttgttttaacagatgccaattacggtgtacagtttaacagagttttctgttgcattaggataaacattaattggagtgcagctaacatgagtatcatcagactagtatcaagtgttctaaaatgaaatatgagaagatcctgtcacaattcttagatctggtgtccagcatggatgaaacctttgagtttggtccctaaatttgcatgaaagcacaaggtaaatattcatttgcttcaggagtttcatgttggatctgtcattatcaaaagtgatcagcaatgaagaactggtcggacaaaatttaacgttgatgtaatggaattccagatgtaggcattccccccaggtcttttcatgtgcagattgcagttctgattcatttgaataaaaaggaacttgg

SEQ ID NO: 3 - DNA, 1138 bp, Homo sapiens
cccttccctgcccgacacccagaccgaccttgaccgcccacctggcaggagcaggacaggacggccggacgcggccatggccgagctcccggggccctttctctgcggggccctgctaggcttcctgtgcctgagtgggctggccgtggaggtgaaggtacccacagagccgctgagcacgcccctggggaagacagccgagctgacctgcacctacagcacgtcggtgggagacagcttcgccctggagtggagctttgtgcagcctgggaaacccatctctgagtcccatccaatcctgtacttcaccaatggccatctgtatccaactggttctaagtcaaagcgggtcagcctgcttcagaacccccccacagtgggggtggccacactgaaactgactgacgtccacccctcagatactggaacctacctctgccaagtcaacaacccaccagatttctacaccaatgggttggggctaatcaaccttactgtgctggttccccccagtaatcccttatgcagtcagagtggacaaacctctgtgggaggctctactgcactgagatgcagctcttccgagggggctcctaagccagtgtacaactgggtgcgtcttggaacttttcctacaccttctcctggcagcatggttcaagatgaggtgtctggccagctcattctcaccaacctctccctgacctcctcgggcacctaccgctgtgtggccaccaaccagatgggcagtgcatcctgtgagctgaccctctctgtgaccgaaccctcccaaggccgagtggccggagctctgattggggtgctcctgggcgtgctgttgctgtcagttgctgcgttctgcctggtcaggttccagaaagagagggggaagaagcccaaggagacatatgggggtagtgaccttcgggaggatgccatcgctcctgggatctctgagcacacttgtatgagggctgattctagcaaggggttcctggaaagaccctcgtctgccagcaccgtgacgaccaccaagtccaagctccctatggtcgtgtgacttctcccgatccctgagggcggtgagggggaatatcaataattaaagtctgtgggtaccaaaaaaaaaaaaaaaaaa

SEQ ID NO: 4 - DNA, 2821 bp, Homo sapiens
gaaaaaagaaaatgtcagaggaatttgaagccaatactatggattctctggtagacatgccatttgctactgtagatattcaggatgactgtggaatcactgatgaacctcaaataaatttgaagagaagtcaagaaaatgaatgggtcaagagtgatcaagtaaagaagaggaaaaaaaagagaaaagattatcaacccaactatttcctgtccattccaatcaccaacaaagagattataaaaggaattaagatcctgcagaatgcaataatacaacaagatgagcgactggccaaagcaatggtcagtgatggttcctttcatattaccctgctggtgatgcaattattaaatgaagatgaagtaaacattggtattgatgctcttttggaattgaaaccattcatagaagaactcctccagggaaaacatttgactttgccctttcaagggattggtacttttggaaatcaggttggatttgtgaagctggcagaaggagatcatgtaaactcacttttggagatagcagagactgcaaataggacatttcaagaaaaaggcatcctggtaggagagagcagaagttttaaacctcatttgaccttcatgaagttgtcaaaatcaccgtggctccgtaagaatggagtgaaaaaaatagatcctgatttatatgaaaagtttatcagtcacagatttggagaagaaatattatatcgcatagatctttgctccatgctgaagaaaaaacaaagtaatggttattatcactgtgaatcttccattgtgattggtgaaaagaacggaggggagcccgatgacgctgaactagtaaggctcagtaagaggctggtggagaacgcggtgctcaaggctgtccagcagtatctggaggaaacacagaataaaaacaagccgggggaggggagctctgtgaaaaccgaagcagctgatcagaatggcaatgacaatgagaacaacaggaaatgagcccggaacgcaggcccccatgtctctgtgcaaagcctccctgcttccctctgctgagtctagggactgacttgcagcgtgctgtttaagttaagtttctctggtgcaatctgtgaagattgcctaatacttttcatgatcgatgtgttcgcattgctgaaacacaacagaagaaaaatggagtgctgggactggcagaggaaattaattgatgaaagaagaatggcccaagtttcattcgccctcagccacgcacaagggaaagggaactttgggttatgcctcctggacgcaaattaaaggccgagaaagaggccttgccatcaatggaatactgccatttatattgcttagcagggcatttgactactttatctgaggccagaactctcacacacagctatcaagtgctaagtttaaaataatcactgttggaattgtcatctgtacaattagtccataatgtttcatgtttgtcctaagtgtgctgttgctatgcagtgtgatctttatttatagtaaattatgtttcatgtaaatgatatatttttggtgaaatgcaaccttttctataaaatgtgggcaacattttaaagtttttttaaaatcctattttgataagtcagtatgccatatttaatgaaatgttattatataatttttttttcttaggcaagaaacctattggaattcgagacttaattaatgaagctttgcatcgagaaacgatgggtctgaagtccaaagtgaaacagataaaggaacttttattaaagcctgagactcaggccagaattaggagggagctttttgaaggaagacttattaacaacagtaattcagcaaatgacgttgatttcagcacaactttgacataagctctacattgcgattgtgacaacatagcttatgaaatcttttcagcttattaagtagctctttggtaaacaccaaagaagtttctgatagtgtctgcacaacagcaaaccaacatttggtgaggaattagcaatttcttgccaaagaaaattgattctgcccaattattttttgagctacacttgtgttttagaatatctgtttctgtaatattgagagttattttatagaaatgatttcttaattagctgttgtgagatatttctcgggtccttgcagaaaaaaacatacagactgtgaacaaatcattcacaaacagaataaaacagagccaacaacagtattttaagggtcacttgcctcctgttgacacaattgttgctaaatcaaaagaagcgttgtccaggtgtgtctacatctagtgttacttttaatgagaatttgaatgtttattgaacaatagtacttgaatgaacatttataaatgtaattattgcgatcactggttaagaatgttttatatatccttataatatttttcactgatcaaaatgttgttctgctttttcatttcttaaggaatacatgtttgggatttttattttttacgtgtccgaagataagctccaggtcttatcgtatcccttgccatctgaacttgtttgcactgcttctgtttgaaagagcatcttgaaaaacttccccggtatgatgattgttggtaacaactttttctatagtcattgatggagtagatcatgatggaggggaaatcactggagatcaaatatgtaaaatcatttcaaatataaaatccagtttactcatggattttagctattttttcactgggtaaattatactacatttatttacaaatgagtttatgcattttcatggctcttaataaacatattgttttcccttgaaaaaaaaaaaaaaaaaaaaaa

SEQ ID NO: 5 - DNA, 1401 bp, Homo sapiens
ccgagtctcaccctcccaggcagctcctacactcaactgcttctctaggaaaggtctcacctccagcctggagcagtcgggattacagaaagccccatccttggcttagggagcgccatgacgactgaaattggttggtggaagctgactttcctccggaaaaagaaatccactcccaaagtgctgtatgagatccctgacacctatgcccaaacagagggagatgcagaacccccgaggcctgacgctggaggccccaacagcgactttaacacccgcctggagaagattgtggacaagagcacaaagggcaagcacgtcaaggtctccaactcaggacgcttcaaggagaagaagaaagtgagagccacgctggcagagaaccctaacctctttgatgatcacgaggaaggacggtcatcaaagtgaagggctgaggagggtgctagcacctcttggctccctgccatcagccagatctgagacaggaccttgccacgctggcctctttggccatagctgaagctgtggggccagttgatacctgctggcaggaaatggctgttttttaggtttgtatttatgtgccgccacttttgtaaggcctgggagatcccagggtcctccaccctccccctgaccacatacaaaggcactctagttcaagagtgaaaaatctcacccaggaggaacagccctccttgaagcaatggcagggccagcagggaggtgggcatggcagggaatggagagagtgagccagacagacttcacctccttactggacacagggtcaagggcgagtttcaattgctgctccctttactttctctacctgtgactactccctggaccaatcctgaggagggcacattttccagaagccacgtgataggggctggtttctgtggagccagaggcagagacactgaacttgagctcacctcctaacaccggcagtaaacttcctggaactttgccctcaggtgcggaggggacagaggaccctggcactctgttagggtgctgtagaagactagattgatggtagtttggcctgttagttcctgttttggccatgacttttgcagatggcaagtcacacaccctcaaagggaagctacacgggccaaatcgggggagtgggtggggaattttctcctctccctttcctactataatagtatttaagacatatcagctccagagatgagtcctggagccttgaattttgtttaacaaaataattgtaggtttctctctgtaataacaacgctggaaaggccgagaacctcttttatgctcatgtcttgcatttattgagatgactgtttctcatgcctttatgttccttcatgtaagtaaagtggacctttgtgctcaaaaaaaaaaaaaaaaaaa

SEQ ID NO: 6 - DNA, 1841 bp, Homo sapiens
agctgggaccggagggtgagcccggcagaggcagagacacacgcggagaggaggagaggctgagggagggaggtggagaaggacgggagaggcagagagaggagacacgcagagacactcaggaggggagagacaccgagacgcagagacactcaggaggggagagacaccgagacgcagagacacccaggccggggagcgcgagggagcgaggcacagacctggctcagcgagcgcggggggcgagccccgagtcccgagagcctgggggcgcgcccagcccgggcgccgaccctcctcccgctcccgcgccctcccctcggcgggcacggtatttttatccgtgcgcgaacagccctcctcctcctctcgccgcacagcccgccgcctgcgcgggggagcccagcacagaccgccgccgggaccccgagtcgcgcaccccagccccaccgcccaccccgcgcgccatggaccccaaggaccgcaagaagatccagttctcggtgcccgcgccccctagccagctcgacccccgccaggtggagatgatccggcgcaggagaccaacgcctgccatgctgttccggctctcagagcactcctcaccagaggaggaagcctccccccaccagagagcctcaggagaggggcaccatctcaagtcgaagagacccaacccctgtgcctacacaccaccttcgctgaaagctgtgcagcgcattgctgagtctcacctgcagtctatcagcaatttgaatgagaaccaggcctcagaggaggaggatgagctgggggagcttcgggagctgggttatccaagagaggaagatgaggaggaagaggaggatgatgaagaagaggaagaagaagaggacagccaggctgaagtcctgaaggtcatcaggcagtctgctgggcaaaagacaacctgtggccagggtctggaagggccctgggagcgcccaccccctctggatgagtccgagagagatggaggctctgaggaccaagtggaagacccagcactaagtgagcctggggaggaacctcagcgcccttccccctctgagcctggcacataggcacccagcctgcatctcccaggaggaagtggaggggacatcgctgttccccagaaacccactctatcctcaccctgttttgtgctcttcccctcgcctgctagggctgcggcttctgacttctagaagactaaggctggtctgtgtttgcttgtttgcccacctttggctgatacccagagaacctgggcacttgctgcctgatgcccacccctgccagtcattcctccattcacccagcgggaggtgggatgtgagacagcccacattggaaaatccagaaaaccgggaacagggatttgcccttcacaattctactccccagatcctctcccctggacacaggagacccacagggcaggaccctaagatctggggaaaggaggtcctgagaaccttgaggtacccttagatccttttctacccactttcctatggaggattccaagtcaccacttctctcaccggcttctaccagggtccaggactaaggcgtttttctccatagcctcaacattttgggaatcttcccttaatcacccttgctcctcctgggtgcctggaagatggactggcagagacctctttgttgcgttttgtgctttgatgccaggaatgccgcctagtttatgtccccggtggggcacacagcggggggcgccaggttttccttgtcccccagctgctctgcccctttccccttcttccctgactccaggcctgaacccctcccgtgctgtaataaatctttgtaaataa

SEQ ID NO: 7 - DNA, 1040 bp, Homo sapiens
accgcggcgcgccgccctccgccgttatatgaggccccgctccggccccacgcggaacccgcggctccgagccttcgccggcgtcccgacccgaggccggacccgaggccagtcccgccgctgcgcagccgaagccagtgcggggcctgagagggacgcgcgccccggggcccccgccgcgggcaccatgggcgctgcccactccgcgtctgaggaggtgcgggagctcgagggcaagaccggcttctcatcggatcagatcgagcagctccatcggagatttaagcagctgagtggagatcagcctaccattcgcaaggagaacttcaacaatgtcccggacctggagctcaaccccatccgatccaaaattgttcgtgccttcttcgacaacaggaacctgcgcaagggacccagtggcctggctgatgagatcaatttcgaggacttcctgaccatcatgtcctacttccggcccatcgacaccaccatggacgaggaacaggtggagctgtcccggaaggagaagctgagatttctgttccacatgtacgactcggacagcgacggccgcatcactctggaagaatatcgaaatgtggtcgaggagctgctgtcgggaaaccctcacatcgagaaggagtccgctcgctccatcgccgacggggccatgatggaggcggccagcgtgtgcatggggcagatggagcctgatcaggtgtacgaggggatcaccttcgaggacttcctgaagatctggcaggggatcgacattgagaccaagatgcacgtccgcttccttaacatggaaaccatggccctctgccactgacccaccgccacctccgcggagaaactgcactttgcaatggggccgcctccccgcgtagctggagcagcccaggcccggcggacagcctcttcctgcagcgccggtacatagccaaggctcgtctgcgcaccttgtgtcttgtagggtatggtatgtgggacttcgctgtttttatctccaataaaaaaaaaaaaaaggtttgttaatt

SEQ ID NO: 8 - DNA, 1119 bp, Homo sapiens
accaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaattta

SEQ ID NO: 9 - DNA, 1444 bp, Homo sapiens
acggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagcc

SEQ ID NO: 10 - DNA, 2101 bp, Homo sapiens
ggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactg

SEQ ID NO: 11 - DNA, 2101 bp, Homo sapiens
gccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctaccctttttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgacttt

SEQ ID NO: 12 - DNA, 3410 bp, Homo sapiens
gaaggggacggggcggccccagtcggaggtcgcagggagctccgcccccgactcggtataagagctgggcccggcccacggcggcggcggcggcggcggagagagctggctcagggcgtccgctaggctcggacgacctgctgagcctcccaaaccgcttccataaggctttgcctttccaacttcagctacagtgttagctaagtttggaaagaaggaaaaaagaaaatccctgggccccttttcttttgttctttgccaaagtcgtcgttgtagtctttttgcccaaggctgttgtgtttttagaggtgctatctccagttccttgcactcctgttaacaagcacctcagcgagagcagcagcagcgatagcagccgcagaagagccagcggggtcgcctagtgtcatgaccagggcgggagatcacaaccgccagagaggatgctgtggatccttggccgactacctgacctctgcaaaattccttctctaccttggtcattctctctctacttggggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaagtggcccagacctcgctggtggtacagaatgtttcagtcatcctgtgtggaatcatcctgatgatggttttcttacataaacatgagcttctgaccatgtaccatggatgggttctcacttcctgctatatcctgatcatcactattgcaaatattgcaaatttggccagtactgctactgcaatcacaatccaaagggattggattgttgttgttgcaggagaagacagaagcaaactagcaaatatgaatgccacaatacgaaggattgaccagttaaccaacatcttagcccccatggctgttggccagattatgacatttggctccccagtcatcggctgtggctttatttcgggatggaacttggtatccatgtgcgtggagtacgttctgctctggaaggtttaccagaaaaccccagctctagctgtgaaagctggtcttaaagaagaggaaactgaattgaaacagctgaatttacacaaagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtctttggtcctttgatttaactgtgacacagttgctgcaagaaaatgtaattgaatctgaaagaggcattataaatggtgtacagaactccatgaactatcttcttgatcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaccacagctgtgccttgagaactaaaagctgtttaggaaacctaagtcagcagaaattaactgattaatttcccttatgttgaggcatggaaaaaaaattggaaaagaaaaactcagtttaaatacggagactataatgataacactgaattcccctatttctcatgagtagatacaatcttacgtaaaagagtggttagtcacgtgaattcagttatcatttgacagattcttatctgtactagaattcagatatgtcagttttctgcaaaactcactcttgttcaagactagctaatttatttttttgcatcttagttatttttaaaaacaaattcttcaagtatgaagactaaattttgataactaatattatccttattgatcctattgatcttaaggtatttacatgtatgtggaaaaacaaaacacttaactagaattctctaataaggtttatggtttagcttaaagagcacctttgtatttttattatcagatggggcaacatattgtatgaagcatatgtagcacttcacagcatggttatcatgtaagctgcaggtagaagcaaagctgtaaagtagatttatcacacaatgactgcatacagacttcaaatatgtcaatagtttggtcatagaacctagaagccaaaagccacacagaagggcaagaatcccaatttaactcatgttatcatcattagtgatctgtgttgtagaacatgagggtgtaagccttcagcctggcaagttacatgtagaaagcccacacttgtgaaggttttgttttacaaatcacttgatttaacacactcaggtagaatatttttatttttactgttttatacccagaagttatttctacattgttctacagcaagaatattcataaaagtatccctttcaaatgcctttgagaagaatagaagaaaaaaagtttgtatatattttaaaaaattgttttaaaagtcagtttgcaacatgtctgtaccaagatggtactttgccttaaccgtttatatgcactttcatggagactgcaatacgttgctatgagcactttctttatccttggagtttaatcctttgcttcatctttctacagtatgacataatgatttgctatgttgtaaaatctttgtaaaaaatttctatataaaaatattttgaaaatctt

(19) United States
(12) Patent Application Publication
(10) Pub. No.: US 2007/0270504 A1
(43) Pub. Date: Nov. 22, 2007

(54) Identification of Therapeutic Agents Using Genetic Fingerprinting

(75) Inventors: Paul E. Young, Sudbury, MA (US); Qin Zong, Germantown, MD (US); Stephen K. Horrigan, Poolesville, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(73) Assignee: AVALON PHARMACEUTICALS, INC., Germantown, MD (US)

(21) Appl. No.: 10/561,372

(22) PCT Filed: Jun. 17, 2004
(86) PCT No.: PCT/US04/19286

§ 371 Date: Apr. 23, 2007

Related U.S. Application Data
(60) Provisional application No. 60/480,013, filed on Jun. 20, 2003.
(60) Provisional application No. 60/517,369, filed on Nov. 5, 2003.

(51) Int. Cl. C12Q 1/68; A61K 45/00; A61P 35/00; C07H 21/02; C07H 21/04; C12N 5/10

(52) U.S. Cl. 514/789; 435/325; 435/6; 536/23.1

(57) ABSTRACT

Methods for identifying compounds having similar biological activity based on similarity in genetic expression profile in cells contacted with one or more of said compounds, along with specifically identified compounds, are disclosed.

This application claims priority of U.S. Provisional Applications Ser. No. 60/480,013, filed 20 Jun. 2003, and 60/517,369, filed 5 Nov. 2003, the disclosures of both of which are hereby incorporated by reference in their entirety.

### FIELD OF THE INVENTION

The present invention relates to biologically active compounds and methods of identifying biologically active compounds based on the activity of such compounds in modulating the expression of a set of genes determined to be modulated by a plurality of compounds exhibiting a common biological activity.

### BACKGROUND OF THE INVENTION

Many different agents are known to possess biological activity, including therapeutic activity, and for many of these the molecular mechanism of action is known. Thus, such compounds may be determined to be related to each other in that they have a common mechanism of action, which mechanism may bear some relationship to the chemical properties of the compounds or to their overall molecular shape. Alternatively, such compounds may not be similar in overall molecular shape or properties but may still, for diverse reasons, operate biologically in a similar manner. In addition, such compounds, related by mechanism of action (MOA) may also show other properties in common and thus these MOA-related sets of compounds may be formed into distinct groups based on their common biological activity. It would be advantageous to be able to take advantage of this relationship based on common MOA by devising screening assays for other compounds having similar biological activity. Because methods of analyzing gene expression are subject to use in large screening assays, where such methods, including rapid measurement of messenger RNA species coupled with methods of reverse transcriptase-polymerase chain reaction amplification for ease of measurement, are susceptible to high degrees of automation, such genetic methods present themselves as a ready medium for high throughput screening for agents having a selected biological activity. The present invention takes advantage of such methods to provide high throughput screening assays (HTSA) capable of rapidly identifying agents having therapeutic activity.

### BRIEF SUMMARY OF THE INVENTION

In one aspect, the present invention relates to a method for identifying a compound having selected biological activity comprising:

(a) contacting a cell with each of a plurality of compounds exhibiting similar biological activity and determining a change in the expression of a plurality of genes of said cell as a result of said contacting whereby the relative changes in expression of said genes together forms a gene expression profile;

(b) contacting a compound different from that of (a) with a cell containing said determined genes of (a) and determining a change in expression of said determined genes as a result of said contacting whereby the relative changes in expression of said determined genes together forms the gene expression profile of (a) thereby identifying a biologically active compound.

In another aspect, the present invention relates to a method for identifying a compound with a selected activity, comprising:

(a) determining a change in the expression profile of a selected set of genes in the presence and absence of a first compound having a desired or selected activity,

(b) determining a change in the expression profile of the selected set of genes of step (a) in the presence and absence of a second compound,

(c) comparing said determined change in expression profile in step (b) with that in step (a)

wherein a determination in step (c) of the same or similar change in said expression profile identifies said second compound as a compound having said selected activity.

In an additional preferred embodiment, the cell of (a) is a colon cell, such as a cancer cell of such organ or tissue.

In one preferred embodiment, the compound of (b) is not an agent possessing known biologically activity so that the methods of the invention find their greatest use in identifying novel agents with a selected biological activity.

The present invention also relates to related gene sets, including those whose polynucleotide sequences correspond to the sequences of SEQ ID NO: 1-12, which gene set is useful in the methods of the invention.

The present invention further relates to compound identified as having biological activity by the methods of the invention. In preferred embodiments, such identified compounds have therapeutic activity, and/or anti-neoplastic activity, and/or enzyme inhibitory, as first determined by the methods disclosed herein.

The present invention also relates to a method for treating a disease comprising administering to an animal afflicted with said disease of a therapeutically effective amount of a compound identified by the methods of the invention as having therapeutic activity. In a preferred embodiment, said therapeutic activity is anti-neoplastic activity.

### DEFINITIONS

As used herein and except as noted otherwise, all terms are defined as given below.

In accordance with the present invention, the term “DNA segment” or “DNA sequence” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the segment and its component nucleotide sequences by standard biochemical methods, for example, using a cloning vector. Such segments are provided in the form of an open reading frame uninterrupted by internal non-translated sequences, or introns, which are typically present in eukaryotic genes. Sequences of non-translated DNA may be present downstream from the open reading frame, where the same do not interfere with manipulation or expression of the coding regions.

The term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

In accordance with the present invention, the term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

The term “primer” means a short nucleic acid sequence that is paired with one strand of DNA and provides a free 3′-OH end at which a DNA polymerase starts synthesis of a deoxyribonucleotide chain.

The term “promoter” means a region of DNA involved in binding of RNA polymerase to initiate transcription. The term “enhancer” refers to a region of DNA that, when present and active, has the effect of increasing expression of a different DNA sequence that is being expressed, thereby increasing the amount of expression product formed from said different DNA sequence.

The term “open reading frame (ORF)” means a series of triplets coding for amino acids without any termination codons and is a sequence (potentially) translatable into protein.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

The term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100[1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared

Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases, or any stretch of polynucleotides that could be synthetically synthesized.

The term “correspond” means that the gene has the indicated nucleotide sequence or that it encodes substantially the same RNA as would be encoded by the indicated sequence, the term “substantially” meaning about at least 90% identical as defined elsewhere herein and includes splice variants thereof.

The term “corresponding genes” refers to genes that encode an RNA that is at least 90% identical, preferably at least 95% identical, most preferably at least 98% identical, and especially identical, to an RNA encoded by one of the nucleotide sequences disclosed herein (i.e., SEQ ID NO: 1-12). Such genes will also encode the same polypeptide sequence as any of the sequences disclosed herein, preferably SEQ ID NO: 1-12, but may include differences in such amino acid sequences where such differences are limited to conservative amino acid substitutions, such as where the same overall three dimensional structure, and thus the same antigenic character, is maintained. Thus, amino acid sequences may be within the scope of the present invention where they react with the same antibodies that react with polypeptides comprising the sequences of SEQ ID NO: 1-12 as disclosed herein.

The term “related gene set” refers to a set of genes, perhaps 5, 10 or more genes, such as those corresponding to the sequences disclosed herein, whose pattern of expression in a cell, expression is modulated by a given set of biologically active agents, especially where said agents exert said activity by a common molecular mechanism.

As used herein, the terms “gene expression profile” or “gene expression fingerprint” are interchangeable and refer to the pattern of gene expression modulation, including increase or decrease of expression, exhibited by an the members of a set of chemical agents with established biological activity when determined using a related gene set. Thus, for a set of 10 genes, possibly genes 1-6 are reduced in expression and genes 7-10 are increased in expression after contact with each of a set of agents having common biological activity. These genes represent a related gene set. The profile or fingerprint will include the relative degree of increase or decrease of expression of the genes of the set in response to the presence of a given concentration of an established biologically active agent (for example, expression of gene 1 may be reduced by half, gene 2 by ⅔, gene 3 not expressed at all, gene 7 doubled in expression, gene 10 increased 3 fold in expression, and so on in response to each of the compounds of the set and relative to the steady state levels of said genes). In the typical case, compound A is introduced into the growth medium of the cells. The result is a gene expression profile, or gene expression fingerprint, or expression fingerprint, for compound A and other compounds of the set possessing common biological activity.

As used herein, the term “compound classifier” refers to a profile of transcriptional changes across a specific set of 10-40 genes that are induced in cells by multiple chemotypes with similar mechanisms of action or biologic function. A compound classifier simply seeks to define unique transcriptional profile associated with a group of related compounds.

As used herein, the term “compound profile” can be generated by the combination of compound classifiers from a diversified reference compound collection. The compound profiler provides a global comparison of compound treatments to a reference compound database. Compound profilers can be effectively used to define and predict a variety of properties of interest.

### DETAILED SUMMARY OF THE INVENTION

In accordance with the present invention, model cellular systems using cell lines, primary cells, or tissue samples are maintained in growth medium and may be treated with compounds at a single concentration or at a range of concentrations. At specific times after treatment, cellular RNAs are isolated from the treated cells, primary cells or tissues, which RNAs are indicative of expression of the different genes. The cellular RNA is then divided and subjected to analysis that detects the presence and/or quantity of specific RNA transcripts, which transcripts may then be amplified for detection purposes using standard methodologies, such as, for example, reverse transcriptase polymerase chain reaction (RT-PCR), etc. The presence or absence, or levels, of specific RNA transcripts are determined from these measurements and a metric derived for the type and degree of response of the sample versus the steady state levels of such transcripts when the compound is not present. The relative levels of RNA transcripts following said contacting with each of a set of agents having established biological activity, including therapeutic activity, such as anti-neoplastic activity, and/or enzyme inhibitory activity and the like serves to define a related gene set and the expression profile of this set provides the fingerprint for the established biologically active agent.

Also in accordance with the present invention, there are disclosed a set of genes and gene sequences whose expression is, or can be, as a result of the methods of the present invention, used to define a related gene set. Thus, the methods of the present invention identify novel therapeutic, including anti-neoplastic, agents based on their exhibiting the same fingerprint as an established biologically active agent (and disclosed herein in specific model systems, such as the HT29 colon cancer cell line). The methods of the invention may be used with a variety of cell lines or with primary samples from tissues maintained in vitro under suitable culture conditions for varying periods of time, or in situ in suitable animal models.

The present invention also provides screening assays for identifying biologically active agents, whether the underlying chemical structures are novel or otherwise, based on the action of such agents to modulate such gene sets in a manner similar to that of an established biologically active agent.

In one highly specific embodiment of the present invention, an established biologically active agent, such as an agent found to inhibit the growth or metastasis of, or kill, cancerous cells, is used to identify a set of cancer related genes by determining the genes present in a cancerous cell whose expression is modulated when said cell is contacted with each of a set of agents having established biological activity, including therapeutic activity, such as anti-neoplastic activity, and/or enzyme inhibitory activity and the like. Thus, as a result of such contacting, genes whose expression changed versus when said contacting does not occur (i.e., the steady state levels of such gene expression), are found to show increased expression may then be grouped as a cancer related gene set.

In a highly specific but non-limiting example, where said biological activity is anti-neoplastic activity, an established anti-neoplastic agent, compound A, is determined to modulate the expression of 10 genes found in a colon cancer cell, such as an adenocarcinoma, whereby genes these genes show a varying pattern of expression following contacting of the cell with compound A. For example, genes 1 to 7 show reduced expression, or non-expression, while genes 8 to 10 show expression, or increased expression, as a result of said contacting. This set of 10 genes thus represents a cancer related gene set as defined herein. Each of said 10 genes may be modulated to a different extent by said established anti-neoplastic agent. For example, expression of gene 1 may be reduced to a level where expression is no longer detected while gene 2 is reduced to half its expression when compound A is not present. The relative levels of expression of each of the genes in the presence and absence of compound A serves to establish an expression pattern, which then represent a mechanism of action of compound A, or is related to, or indicative of, a mechanism of action of compound A. In accordance with the methods disclosed herein, cancer cells (for example, colon cancer cells) containing these genes are subsequently contacted, for example, in vitro, with agents whose anti-neoplastic activity is to be determined and the expression pattern of the genes of this cancer related gene set (defined by expression pattern produced by compound A) following said contacting is determined. As a result of such screening, an additional agent, compound B, is found to modulate this same set of genes, and by the same relative amounts (i.e., the same expression pattern results). Thus, compound B is deemed to be an anti-neoplastic agent and compounds A and B are deemed to act by the same, or similar, mechanism. In a preferred embodiment, the compounds to be screened will be compounds having structural similarity to compound A.

In an alternative example of the foregoing, the related gene set (here, a cancer related gene set) may be independently determined to be involved in the cancerous state without recourse to the modulating ability of any known anti-neoplastic agent. Such cancer related gene set is then utilized as the basis for screening for anti-neoplastic agents de novo, thereby resulting in the identification of Compound A. In one such embodiment, the genes modulated by compound A may represent a subset of the cancer related gene set. The structure of compound A is then utilized as a basis for testing other compounds for anti-neoplastic activity where such other compounds to be tested are of similar chemical structure to compound A (in the same manner as described above).

As disclosed herein, a set of genes is identified that are expressed at varying levels in a cell in response to contact with each of a set of compounds exhibiting a common biological activity, or possessing a similar mechanism of action, including one unrelated to the modulation of said gene set, and said gene set forms a related gene set. Such related gene sets are deemed “fingerprints” for identifying additional agents with a selected biological activity by their ability to modulate such gene sets, and in the same relative amounts, as agents exhibiting said selected biological activity. Thus, the relative modulation of the same gene set acts as a “fingerprint” for other biologically active agents. In accordance with the present invention, such selected biological activity may include therapeutic activity, such as anti-neoplastic activity, and/or enzyme inhibitory activity and the like.

In one embodiment, the present invention related to a method for identifying a compound having selected biological activity comprising:

(a) contacting a cell with each of a plurality of compounds exhibiting similar biological activity and determining a change in the expression of a plurality of genes of said cell as a result of said contacting whereby the relative changes in expression of said genes together forms a gene expression profile;

(b) contacting a compound different from that of (a) with a cell containing said determined genes of (a) and determining a change in expression of said determined genes as a result of said contacting whereby the relative changes in expression of said determined genes together forms the gene expression profile of (a) thereby identifying a biologically active compound.

In another aspect, the present invention relates to a method for identifying a compound with a selected activity, comprising:

(a) determining a change in the expression profile of a selected set of genes in the presence and absence of a first compound having a desired or selected activity,

(b) determining a change in the expression profile of the selected set of genes of step (a) in the presence and absence of a second compound, preferably wherein the second compound is a test compound whose activity is to be determined or compared with that of the first compound,

(c) comparing said determined change in expression profile in step (b) with that in step (a)

wherein a determination in step (c) of the same or similar change in said expression profile identifies said second compound as a compound having said selected activity.

In preferred embodiments, the biological activity may include therapeutic activity, enzyme inhibitory activity, and/or anti-neoplastic activity. In other preferred embodiments, the compounds useful in step (a) (i.e., as a first compound) comprise one or more topoisomerase II inhibitors, especially one or more selected from Camptothecine (S, +), beta-Lapachone, Suramin sodium salt, Aclacinomycin A from Streptomyces galilaeus, Mitoxantrone dihydrochloride, Etoposide, Doxorubicin hydrochloride, Aurintricarboxylic acid, Epirubicin hydrochloride, and m-AMSA hydrochloride.

In and additional preferred embodiment, the cell of (a) is a colon cell, such as a cancer cell of such organ or tissue. The cells utilized in the methods of the invention may also be recombinant cells engineered to express the determined genes, such as one or more genes of a related, or other selected, gene set, including where the recombinant cell does not express the determined genes absent being engineered to do so, such as by genetic engineering.

In one preferred embodiment, the compound of (b) is not an agent possessing known biologically activity so that the methods of the invention find their greatest use in identifying novel agents with a selected biological activity.

The present invention also relates to related gene sets, including those whose polynucleotide sequences correspond to the sequences of SEQ ID NO: 1-12, which gene set is useful in the methods of the invention. These genes have sequences known in the literature and are summarized in Table 1 with reference to their GenBank Accession numbers. Descriptions of the genes are provided in Table 2.

Thus, as a general example, the present invention comprises a method for determining whether a compound functions through a known mechanism of action, comprising:

(a) contacting said compound with a defined cell line;

(b) determining the expression pattern of a defined number of genes of said cell line; and

(c) comparing said expression patterns of (b) with the expression pattern of said defined number of genes of said cell line with at least one reference compound that functions through a known mechanism based on the similarity of the gene expression of said compound and said at least one reference compound.

TABLE 1

| SEQ ID | | | | | | |
|---|---|---|---|---|---|---|
| NO. | probeId | accession | locusLinkId | unigeneId | geneName | genMappId |
| 1 | OG1505 | AA634799 | 26298 | Hs. 182339 | EHF | U54617 |
| 2 | OG1127 | NM_006017 | 8842 | Hs. 112360 | PROM1 | AF027208 |
| 3 | OG798 | NM_014312 | 23584 | Hs. 112377 | CTXL | AI799005 |
| 4 | OG812 | NM_016377 | 9465 | Hs. 12835 | AKAP7 | AF047715 |
| 5 | OG838 | NM_024320 | 79170 | Hs. 36529 | MGC11242 | NM_024320 |
| 6 | OG477 | NM_032192 | 84152 | Hs. 286192 | PPP1R1B | AK024593 |
| 7 | OG1321 | XM_006697 | 54997 | Hs. 18791 | TSC | AA883422 |
| 8 | OG1234 | XM_017384 | 4316 | Hs. 2256 | MMP7 | L22524 |
| 9 | OG892 | XM_030447 | 6319 | Hs. 119597 | SCD | AF097514 |
| 10 | OG252 | XM_032759 | 6678 | Hs. 111779 | SPARC | J03040 |
| 11 | OG922 | XM_043412 | 1026 | Hs. 179665 | CDKN1A | L25610 |
| 12 | OG1551 | XM_047592 | 30061 | Hs. 5944 | SLC11A3 | AF226614 |

TABLE 2

| SEQ | | | |
|---|---|---|---|
| ID | | | ref Seq |
| NO. | gene Description | av Category | Acc |
| 1 | ets homologous factor | Colon differential | |
| 2 | prominin 1 | Up in Epithelial | NM_006017 |
| 3 | cortical thymocyte | Colon differential; TSA | NM_014312 |
| | receptor (X. laevis | regulated | |
| | CTX) like | | |
| 4 | A kinase (PRKA) anchor | plasma membrane | NM_016377 |
| | protein 7 | | |
| 5 | hypothetical protein | W95024 | |
| | MGC11242 | | |
| 6 | protein phosphatase 1, | Up in Epithelial | NM_032192 |
| | regulatory | | |
| | (inhibitor) subunit 1B | | |
| | (dopamine and cAMP | | |
| | regulated phospho- | | |
| | protein, DARPP-32) | | |
| 7 | hypothetical protein | Colon differential_MMC | XM_006697 |
| | FLJ20607 | | |
| 8 | matrix metallopro- | Colon differential; TSA | XM_017384 |
| | teinase 7 (matrilysin, | regulated | |
| | uterine) | | |
| 9 | stearoyl-CoA desaturase | TOX genes down in tox | XM_030447 |
| | (delta-9-desaturase) | | |
| 10 | secreted protein, acidic, | Breast up | XM_032759 |
| | cysteine-rich | | |
| | (osteonectin) | | |
| 11 | cyclin-dependent kinase | cell cycle; | XM_043412 |
| | inhibitor 1A (p21, Cip1) | Oncogenes/TSGs | |
| 12 | solute carrier family 11 | plasma membrane | XM_047592 |
| | (proton-coupled divalent | | |
| | metal ion transporters), | | |
| | member 3 | | |

The nucleotide and amino acid sequences deposited in GenBank, along with ancillary information included therewith, under the accession numbers identified in Tables 1 and 2, are hereby incorporated by reference in their entirety.

The present invention further relates to compounds identified as having biological activity by the methods of the invention. In preferred embodiments, such identified compounds have therapeutic activity, and/or anti-neoplastic activity, and/or enzyme inhibitory, as first determined by the methods disclosed herein.

The present invention also relates to a method for treating a disease comprising administering to an animal afflicted with said disease of a therapeutically effective amount of a compound identified by the methods of the invention as having therapeutic activity. In a preferred embodiment, said therapeutic activity is anti-neoplastic activity.

The present invention further relates to a method for identifying a related gene set, as defined herein, comprising:

contacting a cell with each of a plurality of compounds having common biological activity and determining a change in the expression of a plurality of genes of said cell as a result of said contacting where contacting with each of said plurality of compounds results in the same relative changes of expression of said genes and thereby identifying said genes as a related gene set.

In addition, the invention specifically contemplates the testing of compounds in (b) that were not a known biologically active agents but also encompasses cases where the agent may have been known to have such biological activity in one kind of cell but not others that can be tested using the methods herein. In addition, such known, or suspected, biological activity may have been previously determined to involve a different molecular mechanism that that utilized by the methods of the present invention.

In one highly specific embodiment, the related gene set is a cancer related gene set, identified by the modulation of all of its member genes by a given anti-neoplastic agent. The present invention provides a method of using this set as a fingerprint for other anti-neoplastic agents by the method comprising the steps of:

(a) exposing a known cancerous cell to a chemical agent to be tested for antineoplastic activity;

(b) allowing said chemical agent to modulate the activity of one or more genes present in said cell wherein said genes include or comprise a cancer related gene set, such as the sequences of SEQ ID NO: 1-12, or sequences substantially identical to said sequences, or the complements of any of the foregoing;

(c) determining or detecting the expression of one or more genes of step (b);

(d) comparing the expression of said genes in the presence or absence of exposure to chemical agent;

wherein a difference in expression of all of these genes is indicative of anti-neoplastic activity.

In specific embodiments, this relates to the genes whose sequences correspond to the sequences of SEQ ID NO: 1-12. As used herein, the term “correspond” means that the gene has the indicated nucleotide sequence or that it encodes substantially the same RNA as would be encoded by the indicated sequence, the term “substantially” meaning about at least 90% identical as defined elsewhere herein and includes splice variants thereof.

The sequences disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence or they may be wholly synthetic in origin for purposes of practicing the processes of the invention. Because of the processing that may take place in transforming the initial RNA transcript into the final mRNA, the sequences disclosed herein may represent less than the full genomic sequence. They may also represent sequences derived from ribosomal and transfer RNAs. Consequently, the genes present in the cell (and representing the genomic sequences) and the sequences disclosed herein, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous or normal cells used to determine relative levels of expression because they represent the same sequences or are complementary to RNAs encoded by these genes. Such genes also include different alleles and splice variants that may occur in the cells used in the processes of the invention.

The genes of the invention “correspond to” a polynucleotide having a sequence of SEQ ID NO: 1-12, if the gene encodes an RNA (processed or unprocessed, including naturally occurring splice variants and alleles) that is at least 90% identical, preferably at least 95% identical, most preferably at least 98% identical to, and especially identical.to, an RNA that would be encoded by, or be complementary to, such as by hybridization with, a polynucleotide having the indicated sequence. In addition, genes including sequences at least 90% identical to a sequence selected from SEQ ID NO: 1-12, preferably at least about 95% identical to such a sequence, more preferably at least about 98% identical to such sequence and most preferably comprising such sequence are specifically contemplated by all of the processes of the present invention as being genes that correspond to these sequences. In addition, sequences encoding the same proteins as any of these sequences, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within any of the sequences of SEQ ID NO: 1-12.

In carrying out the foregoing assays, relative biological activity may be ascertained by the extent to which a given chemical agent modulates the expression of genes present in a cell of a particular tissue or organ, such as where said genes are part of the genome of said cell. Thus, a first chemical agent that modulates the expression of the genes of a related gene set, or some other selected gene set, where biological activity is therapeutic activity, to a larger degree than a second chemical agent tested by the assays of the invention is thereby deemed to have higher, or more desirable, or more advantageous, therapeutic activity than said second chemical agent. However, the tested agent is deemed therapeutic if it modulates the same related gene set (i.e., has the same gene expression fingerprint) as an established therapeutic agent, although the extent of such modulation may vary somewhat as to one or more of the genes of said gene set.

The gene expression to be measured is commonly assayed using RNA expression as an indicator. Thus, the greater the level of RNA (messenger RNA) detected the higher the level of expression of the corresponding gene. Thus, gene expression, either absolute or relative, such as here where the expression of several different genes is being quantitatively evaluated and compared in order to establish the gene expression profile of a test compound, for example, the genes of a related gene set as disclosed herein, is determined by the relative expression of the RNAs encoded by the various gene members of the set.

RNA may be isolated from samples in a variety of ways, including lysis and denaturation with a phenolic solution containing a chaotropic agent (e.g., triazol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies.

Normally, prior to applying the processes of the invention, steady state RNA expression levels for the genes, and sets of genes, disclosed herein will have been obtained. It is the steady state level of such expression that is affected by potential biologically active agents as determined herein. Such steady state levels of expression are easily determined by any methods that are sensitive, specific and accurate. Such methods include, but are in no way limited to, real time quantitative polymerase chain reaction (PCR), for example, using a Perkin-Elmer 7700 sequence detection system with gene specific primer probe combinations as designed using any of several commercially available software packages, such as Primer Express software., solid support based hybridization array technology using appropriate internal controls for quantitation, including filter, bead, or microchip based arrays, solid support based hybridization arrays using, for example, chemiluminescent, fluorescent, or electrochemical reaction based detection systems.

The gene expression profiling or fingerprinting of the present invention is used in the same way as chemical and molecular data to identify the compounds of the invention. For example, where an established biologically active agent is known to have a particular gene expression fingerprint as defined herein, the present invention contemplates all chemical agents having said fingerprint, especially where said agent was not previously known or suspected of having the established biological activity activity.

If, for example, an average measurement contains a library of some 50,000 chemical compounds, and genes within the related gene set defined by modulation by compound A, an established biologically active agent, and the genes of the set consistently change their patterns of expression in response to particular chemicals (e.g., 5 of the genes always change expression in a coordinated way, such as down-regulation of one gene within a group of 10) then it always causes the down-regulation of the other 9 specific genes as well and with the same profile or fingerprint as for compound A, then these compounds are identified as biologically active agents for further testing for biological activity, such as in vivo.

The biologically active agents identified by the methods disclosed herein may be useful for therapeutic or research purposes and, when such is the case, they are commonly used in the form of a composition. The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition).

The present invention also relates to recombinant cells engineered to contain intrachromosomally or extrachromosomally one or more genes that together form a related gene set as described herein. Such recombinant cells are genetically engineered (transduced or transformed or transfected) with suitable vectors, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli, lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9, animal cells such as CHO, COS or Bowes melanoma; adenoviruses; plant cells, etc. The selection of an appropriate host is deemed to be within the scope of those skilled in the art from the teachings herein.

Promoter regions can be selected from any desired gene using CAT (chloramphenicol transferase) vectors or other vectors with selectable markers. Two appropriate vectors are pKK232-8 and pCM7. Particular named bacterial promoters include lacd, lacz, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs, such as the genes forming a related gene set as defined herein. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation.

Common methods useful herein are those described in detail in Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

The present invention also relates to a process that comprises a method for producing a product, such as by generating test data to facilitate identification of such product, comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accordance with the foregoing, the present invention relates to a method for producing test data with respect to the biological activity of a compound comprising:

(a) contacting a cell with each of a plurality of compounds exhibiting similar biological activity and determining a change in the expression of a plurality of genes of said cell as a result of said contacting whereby the relative changes in expression of said genes together forms a gene expression profile;

(b) contacting a compound different from that of (a) determined genes of (a) and determining a change in expression of said determined genes as a result of said contacting whereby the relative changes in expression of said determined genes together forms the gene expression profile of (a) thereby identifying a biologically active compound; and

(c) producing test data with respect to the gene modulating activity of said compound based on the gene expression profile indicating biological activity.

In one embodiment of the invention, a compound profiler is generated by the following method:
1) Sort the reference compounds into groups based on supervised and/or unsupervised means:
   a. Supervised method: base upon the similar mechanisms of action or biology function of related reference compounds reported in the literature.
   b. Unsupervised method: based on the similarity of the gene profiles (gene clustering or Fishing) or biological function profiles (go profile).
   c. Combination of the Supervised and Unsupervised method: refined the groups generated by the unsupervised method with the supervised method.
2) Generate compound classifier by identifying a set of genes that changes significantly from a cell line or across several cell lines for a given compound or series of compounds (analogs):
   a. Core genes from single cell type, refined by time and/or dose series experiment.
   b. Core genes from multiple cell types, refined by time and/or dose series experiment.
   c. Core genes that can be used to separate one compound bin from the other bins in the reference database.
   d. Identify a common set of core genes as the compound classifier
3) Combine the compound classifiers from a given reference compound database into a compound profile:
   a. Compute the similarity matrixes of the gene profiles of an unknown compound against the reference compound classifier.
   b. Plot all the similarity matrix scores of the unknown compounds against the classifiers to generate the profile map.
   c. Compare the profile maps between the unknown compound and the reference compounds.

In accordance with the present invention, compound profiles were generated based upon reported mechanisms of action (MOAS) for reference compounds. However, one could attempt to establish predictive compound profiles based around other compound groupings, including those based on such properties as toxicity, selectivity, specific molecular targets and/or in vivo efficacy.

In one such example, a compound profile was generated by the following algorithm:
1. Sort reference compounds into groups based upon MOA reported in literature.
2. Treat cells at 5×IC₅₀, IC₉₀, or 40 mM (maximum concentration). Isolate RNA at specific time points following treatment and perform METS analysis.
3. Check the gene expression profiles within each MOA group and, if necessary, divide the compounds into subclasses (e.g. DNA synthesis inhibitors).
4. Search the genes that uniquely show significantly decreased or increased expression levels for each MOA grouping.
5. Test and validate with test data set.
6. Compare relative gene expression levels of the test sample to the expected gene expression levels of the classifiers.
7. Plot the Pearson correlation coefficient of each classifier against the compound treatment to generate a MOA profile map.
8. Compute the similarity matrix of the MOA profile maps between the unknown compound to define the unknown compound groups.
9. Classify the property of the biological function of the unknown based on the similarity of the MOA profiles between the reference compounds and the unknowns.

Alternative embodiments of the invention include finding the genes that always move in each compound treatment within a class, comparing the compound treatments for a class to all other compounds and determine what distinguishes them, and/or generate multiclass comparisons where genes are identified that are unique for each class

In one such application, for every compound in the data set the genes that changed expression level upon chemical treatment is identified. This can be done by comparing the expression level to the expression level of a negative control compound treatment. This is typically vehicle treated cells, but could be an inactive compound or no treatment. Compounds are divided into classes based on known mechanisms of action, and unsupervised expression analysis to identify compounds which act in a similar manner.

In accordance with the foregoing, all compounds in a class are analyzed to identify single genes or combinations of genes that behave in similar way in all samples within the class. This identifies a signature from the class, but makes no assumptions about it being unique compared to other classes. Compare the expression of compound treatments with a class to all other treatments in the database. This allows the identification of patterns of genes that uniquely define each class from all others. This gives what is unique to each class but is dependent of the completeness of the database. A multiclass discriminator would identify those gene sets that are unique to each class and don't overlap another class.

Such comparisons can be done in several ways:
1) T-test based analysis where each gene is tested for difference in the populations
2) Nonparametric approaches like SAM, where no assumtions are made about distributions as in 1
3) Baysian approaches which use the probabilities of the differences
4) Combinatorial gene changes were sets of genes are used together, such as “if gene A and Gene B changes it is class 1, but if only one changes it is class 2.”

In one such example, nonlinear associations include cases such as where a given gene is high or low and is placed in class 1, while no change results in class 2.

It should be cautioned that, in carrying out the procedures of the present invention as disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art. The following example shows how a potential anti-neoplastic agent may be identified using one or more of the genes disclosed herein.

### EXAMPLE

HT29 cells are grown to a density of 10⁵ cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1× PCR buffer, RNAsin, reverse transcriptase, nucleoside triphosphates, amplitaq gold, Tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perkin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference genes is then calculated and a relative expression value determined for all of the samples used. This procedure is then repeated for each of the target genes in a given signature, or characteristic, set and the relative expression ratios for each pair of genes is determined (i.e., a ratio of expression is determined for each target gene versus each of the other genes for which expression is measured, where each gene's absolute expression is determined relative to the reference gene for each compound, or chemical agent, to be screened). The samples are then scored and ranked according to the degree of alteration of the expression profile in the treated samples relative to the control. The overall expression of the set of genes relative to the controls, as modulated by one chemical agent relative to another, is also ascertained. Chemical agents having the most effect on a given gene, or set of genes, are considered the most anti-neoplastic.

What is claimed is:

1. A method for identifying a compound with a selected activity, comprising:
(a) determining a change in the expression profile of a selected set of genes in the presence and absence of a first compound having a selected activity,
(b) determining a change in the expression profile of the selected set of genes of step (a) in the presence and absence of a second compound,
(c) comparing said determined change in expression profile in step (b) with that in step (a)
wherein a determination in step (c) of the same or similar change in said expression profile identifies said second compound as a compound having said selected activity.

2. The method of claim 1 wherein said selected activity is antineoplastic activity.

3. The method of claim 1 wherein said selected set of genes is present in a cell.

4. The method of claim 1 wherein said expression is transcription.

5. The method of claim 1 wherein said change in expression profile is determined by determining synthesis of RNA.

6. The method of claim 1 wherein said change in expression profile is determined by determining polypeptide synthesis.

7. The method of claim 1 wherein said selected activity is inducing a physiological change in a cell.

8. The method of claim 1 wherein said selected activity is therapeutic activity.

9. The method of claim 1 wherein said selected activity is enzyme inhibitory activity.

10. The method of claim 1 wherein the compound of step (a) is a topoisomerase II inhibitor.

11. The method of claim 1 wherein the compound of step (a) is a member selected from the group consisting of Camptothecine (S, +), beta-Lapachone, Suramin sodium salt, Aclacinomycin A from Streptomyces galilaeus, Mitoxantrone dihydrochloride, Etoposide, Doxorubicin hydrochloride, Aurintricarboxylic acid, Epirubicin hydrochloride, and m-AMSA hydrochloride.

12. The method of claim 3 wherein the cell is a colon cell.

13. The method of claim 12 wherein said cell is a cancer cell.

14. The method of claim 3 wherein the cell is a recombinant cell engineered to express said selected set of genes.

15. The method of claim 14 wherein said recombinant cell does not express said selected set of genes absent said engineering.

16. The method of claim 3 wherein said selected set of genes is part of the genome of said cell.

17. A related gene set comprising genes whose polynucleotide sequences correspond to the sequences of SEQ ID NO: 1-12.

18. The method of claim 1 wherein said determined genes are in the gene set of claim 17.

19. A compound identified as having therapeutic activity by the method of claim 1.

20. A compound identified as having anti-neoplastic activity by the method of claim 1.

21. A compound identified as having enzyme inhibitory activity by the method of claim 1.

22. A method for treating a disease comprising administering to an animal afflicted with said disease of a therapeutically effective amount of the compound of claim 19.

23. A method for treating cancer comprising administering to an animal afflicted with cancer of a therapeutically effective amount of the compound of claim 20.

24. A method for identifying a related gene set comprising:
contacting a cell with each of a plurality of compounds having common biological activity and determining a change in the expression of a plurality of genes of said cell as a result of said contacting where contacting with each of said plurality of compounds results in the same relative changes of expression of said genes and thereby identifying said genes as a related gene set.

25. The method of claim 24 wherein said biological activity is therapeutic activity.

26. The method of claim 24 wherein said biological activity is enzyme inhibitory activity.

27. The method of claim 24 wherein said biological activity is anti-neoplastic activity.

28. The method of claim 24 wherein said plurality of compounds are topoisomerase II inhibitors.

29. The method of claim 24 wherein said plurality of compounds comprise members selected from the group consisting of Camptothecine (S, +), beta-Lapachone, Suramin sodium salt, Aclacinomycin A from Streptomyces galilaeus, Mitoxantrone dihydrochloride, Etoposide, Doxorubicin hydrochloride, Aurintricarboxylic acid, Epirubicin hydrochloride, and m-AMSA hydrochloride.

30. A method for producing test data with respect to a biological activity of a compound comprising:
(a) contacting a cell with each of a plurality of compounds exhibiting similar biological activity and determining a change in the expression of a plurality of genes of said cell as a result of said contacting whereby the relative changes in expression of said genes together forms a gene expression profile;
(b) contacting a compound different from that of (a) with the determined genes of (a) and determining a change in expression of said determined genes as a result of said contacting whereby the relative changes in expression of said determined genes together forms the gene expression profile of (a) thereby identifying a biologically active compound; and
(c) producing test data with respect to the gene modulating activity of said compound based on the gene expression profile indicating biological activity.

31. A recombinant cell expressing a related gene set identified by the method of claim 24.

32. A recombinant cell expressing the related gene set of claim 17.
